(19) United States
(12) Patent Application Publication
(10) Pub. No.: US 2013/0303860 A1
(43) Pub. Date: Nov. 14, 2013

(54) SYSTEMS AND METHODS FOR USE IN FALL RISK ASSESSMENT

(71) Applicants: Robert Bender, Ottawa (CA); Trevor Jones, Ancaster (CA); Wendy Porter, Mississauga (CA)

(72) Inventors: Robert Bender, Ottawa (CA); Trevor Jones, Ancaster (CA); Wendy Porter, Mississauga (CA)

(21) Appl. No.: 13/684,097

(22) Filed: Nov. 21, 2012

Related U.S. Application Data
(60) Provisional application No. 61/562,148, filed on Nov. 21, 2011.

(51) Int. Cl. A61B 5/11

(52) U.S. Cl. CPC A61B 5/11
USPC 600/300

(57) ABSTRACT

Systems and methods for use in patient fall risk assessment are provided. In the fall risk assessment system, a Protocol including one or more motion-based tests is performed by a patient. The patient's performance is recorded using one or more sensors and the patient's data is compared to normative data to assess risk within the multifactorial problem of fall risk assessment and management.

### CROSS-REFERENCE TO RELATED APPLICATIONS

The present application claims the benefit of U.S. Provisional Application No. 61/562,148 filed Nov. 21, 2011 and entitled “Method For Assessment Of Fall Risk.”

### BACKGROUND OF THE INVENTION

The present invention generally relates to assessing fall risk. More particularly, the present invention relates to a assessing fall risk for the purpose of improving patient safety in acute and long term care settings.

Patient falls are a persistent, widely occurring problem in hospitals, nursing homes and other institutional settings. Consequently, prevention or minimization of the number of falls is important for many reasons such as preventing additional patient injury and reducing cost and pain due to treatment for additional injury.

When attempting to assess the fall risk for a particular individual, it may be recognized that the individual's fall risk is typically the result of several factors which may be independent or interdependent and which may vary as to the strength of their correlation with fall risk for a particular individual in a particular situation. Some factors, such as environmental factors (such as slippery floors, etc.) may be easily controlled and are typically external to the individual. Conversely, other factors may be internal to the individual. For example, the influence of medications, patient condition, etc. are variable as to circumstances and individuals. As a result, fall risk for and individual may change with time and with the presence or absence of various factors, both internal and external to the patient.

### BRIEF SUMMARY OF THE INVENTION

One or more of the embodiments of the present invention provide methods and systems for use in assessing fall risk for the purposes of assisting efforts to prevent or lower the incidence of patient falls. In the present system, motion-based tasks are performed by a patient. The patient's performance is recorded using one or more sensors and the patient's data is analyzed and compared to one or more indicators in order to quantify a fall risk determination. In one embodiment, the patient's data is compared to normative data to identify increased fall risk. When the patient's performance departs from the normative data, an increased risk of a fall may be identified and recommendations for fall prevention may be made.

### BRIEF DESCRIPTION OF THE DRAWINGS

FIG. 1 illustrates a fall risk assessment system according to an embodiment of the present invention.

FIG. 2 shows a graph of the total force (F total) observed using a balance plate for a fit middle-aged female (A) subject performing a sit-to-stand task

FIG. 3 illustrates an alternative embodiment of the fall risk assessment system.

FIG. 4 illustrates an additional alternative embodiment of the fall risk assessment system.

FIG. 5 illustrates another alternative embodiment of the fall risk assessment system.

FIG. 6 shows a graph of the total force (F total) observed using a balance plate for healthy 86 year old female (B) performing the same sit-to-stand task as in FIG. 2.

FIG. 7 shows graphs of the total force (Ftotal) observed using a force plate for a 53 year old female subject (A), 76 year old female subject (C), and 86 year old female subject (B) performing a sit-to-stand task.

### DETAILED DESCRIPTION OF THE INVENTION

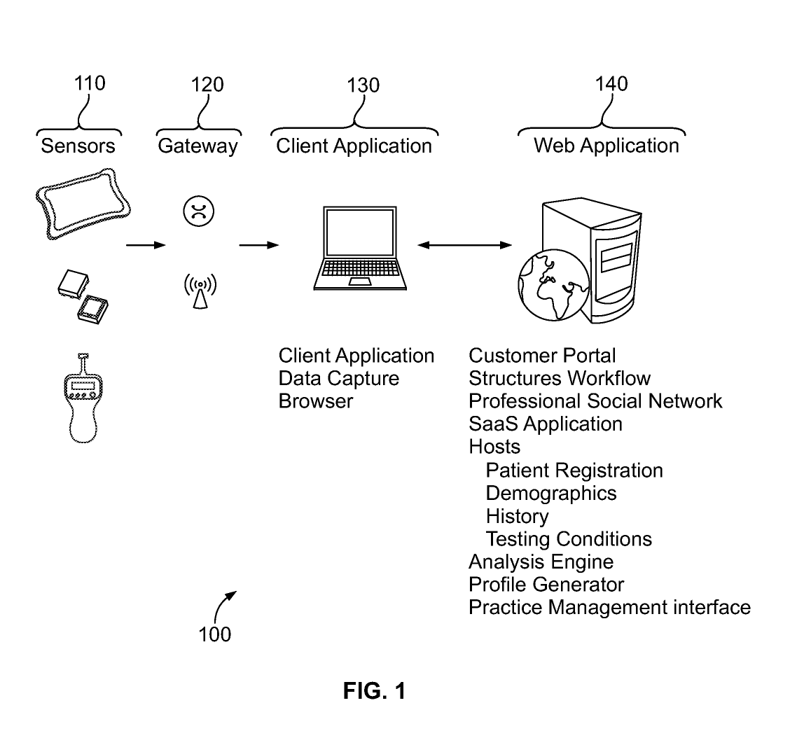

FIG. 1 illustrates a fall risk evaluation system 100 according to an embodiment of the present invention. The fall risk evaluation system 100 includes one or more sensors 110, a gateway 120, a client application 130, and a web application 140.

As further described below, in one embodiment a client or patient is positioned so that the sensor 110 is able to sense and record the motion of the patient. The patient is then instructed to perform a predetermined motion task, such as a sit-to-stand task that requires the patient to stand up from a seated position. The sensor 110 records the patient's motion during performance of the task. The data recorded by the sensor 110 may then be passed to the gateway 120 which relays the data to the client application 130.

At the client application 130, the sensor data may be captured and may also be displayed to an operator, preferably through a browser-driven interface installed as part of the client application 130. Additionally, the sensor data may then be passed from the client application 130 to the web application 140.

As further described below, the web application 140 is preferably located at a centralized location and receives information from numerous client applications. As further described below, the web application 140 preferably includes a customer portal, structures workflow, includes a professional social network, includes a Software-As-A-Service application, hosts databases for patient registration, demographics, history, and testing conditions, includes an analysis engine, includes a profile generator, and includes a practice management interface.

Additionally, one or more embodiments provide a system and method of use which may provide assessment of fall risk. In one embodiment, a subject (S) (such as a patient) interacts with a dynamic measurement device or devices (D_{1 . . . n}). Such measurement devices may include but are not restricted to force or balance plates, accelerometers, inertial measurement units, video capture systems and other sensor systems which may provide data on position, force, rate of motion or other physical results of musculoskeletal action. In general such systems are preferably capable of acquiring data at rates equal to or greater than the repetition rate of the task being measured. Additionally, such systems preferably provide data points at a rate equal to or greater than the rate of involuntary muscular tremor, for example as a rate of 2 per second or higher or 5 per second or 10 per second.

Without limiting generality, further descriptions may refer to force measurement embodiments but dynamic data on either force or position or rate of motion may be used in subsequent analytical steps. Specific embodiments may use low-cost robust consumer products as sensors and computers networked through the internet. A practical consequence of this is portability and ease-of-use.

Additionally, a dynamic measurement device which may explicitly combine several sensor and modalities may be used to yield a time-series of data that may be correlated for the purpose of improving the analysis of fall risk.

This data is captured in a file by use of an interface device such as client application 130 that also may control the sensor device within a local feedback loop. The data file is annotated (tagged) as to the subject and/or patient identity and test performance particulars (start time, end time and general description) by a therapist or operator through the interface. The client application 130 may be a terminal, laptop or mobile device such as a tablet or smartphone.

With respect to FIG. 1, the apparatus and its method of use are broadly described. The description is intended to illustrate the flow and management of data within a network. Internal traffic of signals for internal error correction and security (encryption) are not shown, but are preferably present. For example, the communication of the data from the sensors to the gateway, from the gateway to the client application and/or from the client application to the web application may be encrypted—as may be the communication from the web application to the client application.

Similarly, it is understood that linkages between elements of the apparatus may be hard-wired or wireless and that such elements may be co-located or distributed geographically. The network aspects of the apparatus are a preferred aspect of its functioning. It is common practice in networks to distribute ‘intelligence’ and storage elements in order to optimize performance and such elements as would commonly form part of a network are included. Accordingly, a network embodiment may have several forms and include additional data storage or relay elements.

The specific test (or set of tests) is part of a test library data base which provides the test-related Protocol to which the data is referenced. Protocols may include simple or multiple tests and constrain performance as to time and expected range of performance. In another embodiment of the invention, tests within Protocols may be modified or perturbed so as to elicit performance changes relevant to maintenance of balance. A Protocol is externally defined and not modified by the system except as to duration and/or number of repetitions.

In a practical setting, the fall risk evaluation system which is the subject of FIG. 1 is used by an operator or a therapist working with a subject/patient according to FIG. 1. That is, once a Protocol is selected and initiated (by the operator), the system prompts (or specifies) the specified test or tests in sequence and then, through use of an appropriate set of sensors, test-related performance data is acquired through the gateway 120 which is recorded and transmitted for analysis, feedback and reporting. Optionally, the data acquisition may be directly linked to a display interface to provide immediate confirmation of measurement and performance feedback.

The client application 130 includes an interface device which may have a display and data entry capability so that the user (therapist) may enter relevant annotation and view data as confirmation of task performance.

The data acquired in task-measurement is then transmitted by a hard or wireless link to a server including the web application 140 which hosts an analysis module, and one or more data bases where, using standard methods of mathematical analysis (signal extraction, averaging and statistical processing), relevant performance information may be extracted as further described below.

The server may also link to cloud-based or other servers as is common in data processing networks and thus may access additional data such as patient medical records, demographic or other data stored in independent repositories of such data.

A feature of an embodiment is that data from individual subjects and tests is analyzed on-line with data accumulated from other test subjects and tests thus enabling quantitative comparisons of performance; such comparisons may be to normative populations or over time and also within an individual's performance history.

For illustrative purposes and not restricting generality, an example of a Protocol to assess fall risk and its use is now described. The Protocol starts with the recognition that most falls are from standing posture or from attempts to transition to standing posture and that the ability to change posture (smoothly) is related or correlated to the ability to avoid falling. Accordingly, characterization of the mechanical process involved in a defined postural change (as an example, a sit-to-stand postural change) may provide insight into fall risk. For example, a subject unable to complete a sit-to-stand without assistance has a high fall risk and is a poor candidate for stable ambulation.

Particularly, it has been shown that there are qualitative changes in the observed force vs time relationship as a seated subject moves to a standing position on a balance plate; consequently, a simple sit-to-stand test may be used as an objective clinical monitoring tool for patients who are at risk for falls—particularly as they move from beds in a voluntary sequence which typically begins with a similar motion sequence (sit to stand).

One exemplary Protocol to assess fall risk is now described. First, a subject lying at rest in a bed is instructed to move to a seated position (or with any subject starting at a seated position) and then attempt (unassisted) to rise to a full stand on a balance board (for example, a Nintendo Wii balance board). Measurements of the total force exerted on the balance board are taken during the sit-to-stand process. The measurement data from the balance board typically defines a complex curve, which may be smoothed or subjected to further analysis as to high frequency components which may be indicative of (muscle) tremor. This data is then compared to a demographically relevant sample in a database which has been previously populated and may be compared to longitudinal (time series) data on the subject in order to assess relative fall risk.

Preferred embodiments of the invention include those in which the data is analyzed with explicit or implicit reference to similar data within a data repository. Methods of data analysis include (without limitation) signal averaging and integration, statistical cluster analysis, frequency spectrum analysis, etc. Pre-processing of signal streams is generally preferred for interpretation and feedback; raw force and rate data from sensor systems is not typically directly interpretable.

Accurate quantitation is preferable for assessment and is not available through manual methods due to factors including lack of operator consistency, distractions and the complex interaction of factors involved in test performance. In addition observed force vs time relationships are in a time domain (sub second) not readily assessed by manual methods.

One or more embodiments of the present system do not require an explicit biomechanical model since an aspect of the invention may be the restriction of performance to a defined task at one time. Many such tasks may be utilized singly or in combination and it is a feature of the method that although constrained, the measured variables (eg. force, displacement, velocity) are not predicted from the task definition. According to one or more embodiments of the present invention, analysis of the data obtained from a given performance need not be explicitly related to functional aspects of performance but rather may be carried out by correlation or pattern matching methods. The use of a specific test (Protocol) provides an implicit biomechanical frame of reference so that data between individuals or longitudinal (time) measurements on the same individual may be compared.

In the practice of one or more embodiments of the invention, a subject or patient's interaction with the apparatus is mediated by an operator or therapist who typically but not necessarily provides additional guidance during the interaction. For example, with respect to placement of measurement apparatus relative to the subject, and mitigation of immediate fall risk by physical restraint.

As a typical use is described, it is noted that the apparatus and method of its use is not restricted to a particular measurement or sensor type nor is it restricted to measurement and assessment of a particular task or class of subject. Specific examples of use are given for illustration of the general utility and not intended to be limiting.

In a particular instance of the apparatus, a single or multiplicity of sensors may be used simultaneously to gather data on more than one performance variable. This is illustrated in FIG. 1 where the term sensor refers to any of several single device nodes which may contain multiple transduction elements (such as balance board with a multiplicity of load cells for the purpose of measuring attributes of stance such as lateral asymmetry).

Thus in a particular instance of use, a sensor device may be a balance board which in turn contains several force transducers so that variations of force in an horizontal plane may be measured when a subject is standing on the sensor device 110 of FIG. 1.

The sensor device (balance board) 110 preferably pre-conditions data and produces digital data which are transmitted directly or through an internal radio link to a gateway device 120 which may be a laptop computer but is more typically a data buffer and relay that may act as a local hub for multiple sensors and a local display unit which provides feedback to the subject. This display unit is linked to the main apparatus but need not be directly linked to the local buffer. The display unit may provide performance guidance with respect to the specific task on which measurements are being made.

Within fall risk assessment the following example is illustrative of the utility and functioning of system of FIG. 1. It is known that fall risk generally increases with age and concomitant loss of muscular strength.

Thus, expected outcome measures include a shorter time to complete a sit-to-stand sequence and less ‘performance jitter’ within the approach to the end state. Elderly subjects consistently took longer to complete and show considerably more ‘jitter’ as evidenced by relatively high frequency components in the overall force-time curves.

Consequently, the following protocol may be employed. As discussed above, implementation of the protocol may include displaying instructions to the operator at the client application 130. These instructions may include: ‘Ask the patient to sit sideways on the bed and place (bare) feed on the balance board; then ask the patient to rise unassisted to a full stand and hold for up to 30 seconds (although other time periods may be employed such as 3 s, 5 s, 10 s, 15 s, or 25 s). Then tell the patient that, “following a “beep”, they will have to close their eyes for 30 seconds and maintain stance’ (although other time periods may be employed such as 3 s, 5 s, 10 s, 15 s, or 25 s).

During performance of the Protocol at least the following data are preferably collected: Total force (weight); Force top right (FTR); Force top left (FTL); Force bottom right (FBR); Force bottom left (FBL). Again, in the present example, the sensor employed provides force measurements near the corners of the balance board.

Analysis of measured data in relation to fall risk estimation may be as simple as determining time to task completion; however as will be clear to persons skilled in the art, the opportunity to simultaneously measure, record and compare multiple variables may provide significant improvement in the ability to discriminate performance capability of any complex system. Accordingly, and within the scope of the present invention, multiple measurements are made for the purpose of analysis which is carried out both within specific test data and by comparison (using established statistical or analytical techniques) with aggregated data from other subjects where comparable fall history data is available thereby allowing the development of probabilistic assessment tools for fall risk measurement.

In one embodiment of the present invention, data acquisition may be done in local loop mode wherein the test Protocols guiding performance are downloaded to the local gateway (laptop) and the data stored locally for subsequent transmission to the main server.

One feature of the system is that performance measurement is related to specific tests (Protocols) that define and constrain performance and thus the subject interface (typically a screen display of graphical information) may be driven directly from the local buffer or from the main processer/server.

The main server or web application 140 receives data from the sensors through the gateway 120 and is linked to one or more data repositories (such as data bases). It is may also be linked to a ‘task library’ which comprises a number of specific performance tasks which are used to guide/drive the test process and which are specified by an operator or therapist. The test library (instructions) is a component of the method as it provides biomechanical constraints for data interpretation. One or more display devices may be driven by the main server for the purposes of display of data acquisition and interpretation. These displays may be linked to the server 140 through the internet and browser driven so that the display is independent of device specific constraints.

This allows a therapist to have local access in ‘real time’ for feedback as well as ‘off line’. Additionally the main server or a complex of servers provides the computational resources necessary for data analysis (including analysis in reference to other performance data either from population data bases or longitudinal (time) subject data or both. Such data may be combined for analysis with other data sets such as medical record or other information not necessarily related to specific task performance. Reports generated may be transmitted to other relevant systems across standard application interfaces.

In one embodiment of the system of FIG. 1, a sensor system comprises a balance board (eg. Nintendo Wii); this sensor device includes force transducers and integrated on-board signal processing, digitization and a Bluetooth radio link. The radio signal may be received by a compatible Bluetooth radio device which, in turn may utilize a standard USB port on a conventional laptop computer device. Such a Bluetooth-equipped laptop may function as a gateway for the purposes of the present system and preferably has an integrated display suitable for presentation of test performance data and instructions when driven by suitable software. The software may be configured so as to provide graphic confirmation of test performance using displays of data or symbolic icons or text as desired. Any sensor-gateway link, wired or wireless may be used in the practice of the present system.

In another embodiment of the system of FIG. 1, a sensor system may comprise one or more of an accelerometer or an IMU (such as a device combining an accelerometer, a gyroscopic sensor and a magnetometer) all of which are in common use in current biomechanical measurement practice. Similarly, positional inputs may be measured by video-based sensor systems. Novel measurement devices may be used without limitation in the practice of the invention.

FIG. 3 illustrates an alternative embodiment of a fall risk evaluation system 300 which includes one or more sensors 310, a transmission network 320, a server 330, a local display 340, and a local interface 350. The sensor 310 may be any of the sensors discussed above. In the embodiment of FIG. 3, the data from the sensor passes through a transmission network 320 to the server 330. For example, the sensor 310 may be a web-enabled device that may be configured for plug-and-play integration into a standard web connection. When activated, the sensor 310 may transmit its data directly to a remote server 330 through the transmission network of the internet 320. Additionally, the communications from the sensor 310 to the server 330 may be secured through the use of passwords and/or encryption.

Once the data is received at the server 330, it may be processed as described herein. A graphical display may also be sent from the server to a local display 340 through the transmission network 320 such as the internet. The local display 340 may for example be a screen on a device that may provide a display viewable by the operator and/or patient.

Additionally, the server 330 may communicate with the local interface 350. The local interface may also control the sensor 310. For example, the operator may use the local interface 350 to initiate a protocol for a sensor. Once the sensor 310 records the data and transmits it to the server 330, the server 330 may analyze the data and transmit a command, instruction, or warning to the local interface. For example, the server 330 may request that the protocol be repeated or may identify a different protocol to be employed, or may provide another instruction to the operator with regard to how to position the patient or to provide instructions to the client. Additionally, based on an analysis of the data, the server 330 may send questions and/or instructions to the local interface for the operator to perform. For example, one such question might be “Have you noticed a decline in your ability to do (a specific task)” or “How long have you been experiencing problems doing (a specific task)” or “Do you feel discomfort/strain in any of your knee, hip, ankle, calf, quad, hamstring, gluteus, lower back, stomach? If so, please specify”. The interface 350 also includes an input that allows the operator to indicate the patient's selection. Such an input may include a keyboard, touchscreen, voice commands or buttons, for example.

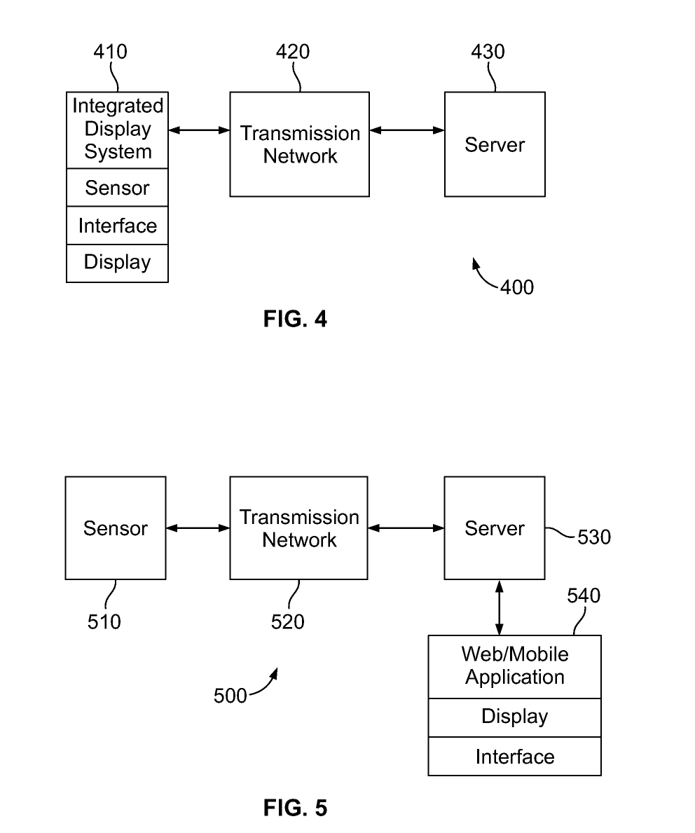

FIG. 4 illustrates an additional alternative embodiment of a fall risk assessment system 400 which includes an integrated display system 410, a transmission network 420, a server 430. The embodiment of FIG. 4 is similar to that of FIG. 3, but the sensor 310, local interface 350, and local display 340 of FIG. 3 are replaced with an integrated display system 410. The integrated display system 410 is preferably a single system wherein the system, interface, and display components are directly connected through a wired or wireless link.

In operation, the interface of the integrated display system 410 is used to activate the sensor which then records data and transmits it to the server 430 through the transmission network 420. Once the server processes the data, display data may be transmitted back through the transmission network 420 to the integrated display system 410 for display on the integrated display system's display. Additionally, the server 430 may send queries, interact with, and receive data and/or commands from the integrated display system's interface.

FIG. 5 illustrates another alternative embodiment of a fall risk evaluation system 500 which includes one or more sensors 510, a transmission network 520, a server 530, and a web/mobile application 540. The embodiment of FIG. 5 is similar to that of FIG. 3, but the sensor 510 is in bi-directional communication with the server 530 through the transmission network 520. Additionally, the web/mobile application 540 is also in bi-directional communication with the server 530. For example, the web/mobile application 540 may communicate with the server through the cellular phone system or through the internet.

In operation, an account may be created at the server 530 which may be password and/or encryption protected. The account may then be associated with one or more sensors 510 and the web/mobile application 540 which may be hosted, for example, on smartphone or a tablet computer such as the iPad®. Additionally, the account may be available through a web browser and may consequently be accessed on any web-enabled device such as a desktop, laptop, tablet, and/or smartphone.

Additionally, the server 530 may establish a premises account associating one or more sensors with one or more web/mobile applications.

In operation, once the sensor and web/mobile application have been associated at the server, the operator may position the patient with regard to the sensor and then initiate the testing protocol using a testing initiation command entered through the interface of the web/mobile application. The testing initiation command may then be transmitted to the server 530 which may identify the associated sensor and then send a command to the sensor 510 through the transmission network 520 in order to activate the sensor. When more than one sensor 510 is present, the web/mobile application 540 includes on its interface a selector for selecting the desired sensor to initiate.

Turning now to specific measurements, in general there are multiple options for measurement of position, force and rate of change of the elements of the musculo-skeletal system and inputs from such measurement subsystems may be utilized in the practice of the present system

A novel and unexpected result of the present system is that assessments of fall risk, when associated with defined tasks, are informative without explicit reference to the specific task. Some performance outputs reflect the results of fundamental functional processes which are convoluted with the signals of a defined task performance. As specific, but not limiting examples:

First, the force vs time (frequency) components at higher (for example, greater than 2 Hz, 5 Hz, or 10 Hz) frequencies are informative as to involuntary processes including pathological tremors and loss of strength. The presence, frequency, and/or amplitude of these involuntary processes are correlated to an increased fall risk.

Second, the displacement around the center of mass (measured by changes in the center of balance or by an IMU placed near the center of mass or by video analysis of upper body motion) is informative as to the ability to maintain balance, and is consequently also correlated with an increased fall risk.

Third, the rate of rise is significantly informative of general fitness and thus inversely correlates with fall risk.

Accordingly, a novel utility of the present system is the characterization of fall risk as reflected by aspects of musculoskeletal task performance using suites of specific defined tasks. A single task-associated variable such as time-to-rise or rate of rise may be combined with one or more other measureable variables to define a region in n-space that may define ‘fall risk’. Quantitative measurement of mechanical outputs associated with the defined tasks allows comparison (correlative or explicit) with population results and or time series from the same subject. These comparisons create profiles within areas of performance (such as frequency domains or differential motion) that are associated with either competence or high risk of falling.

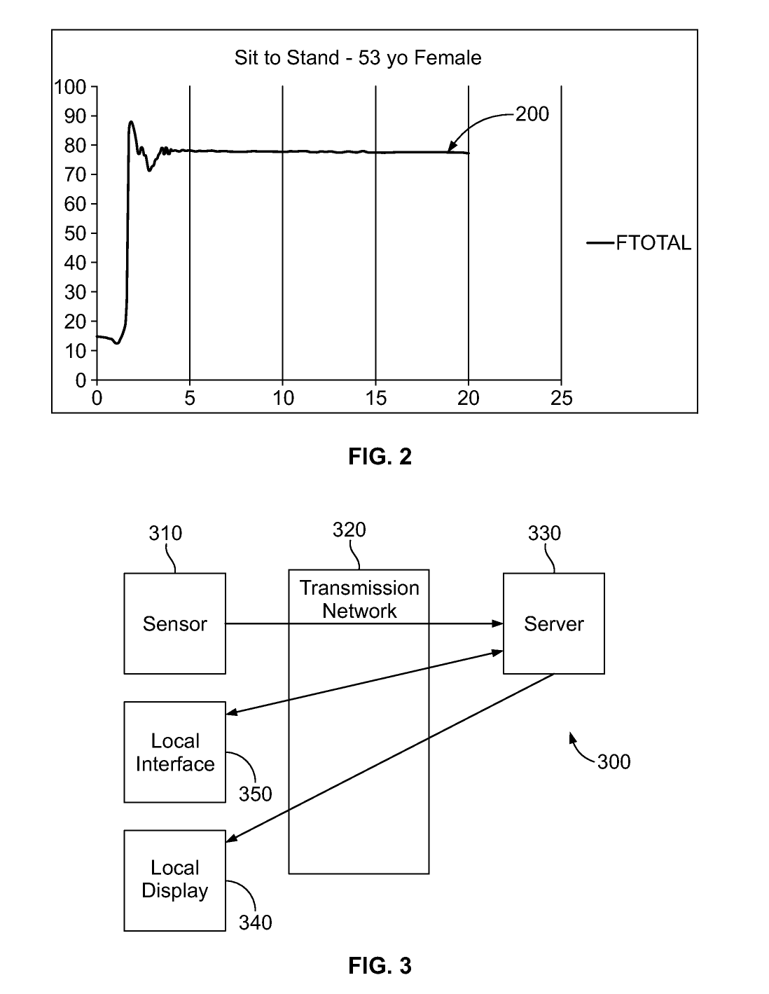

FIGS. 2 and 6-8 below are examples which are illustrative but not limiting which display force vs time charts for a number of specific tasks as performed by several different subjects. Attention is drawn to specific qualitative and semi-quantitative features of these graphs to illustrate how various analytical methods may be applied with respect to both general and specific performance features.

FIG. 2 shows a graph 200 of the total force (F total) observed using a balance plate for a fit middle-aged female (A) subject performing a sit-to-stand task. The curve shown in FIG. 2 is illustrative of a situation of a person at little to no risk of fall based on their physical factors.

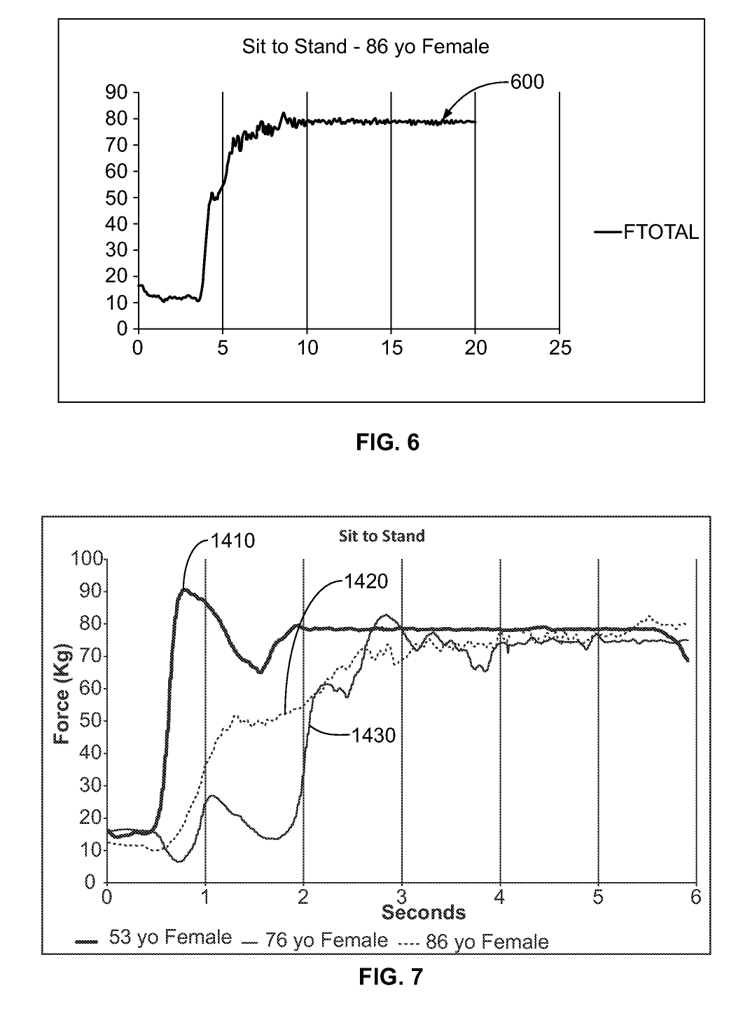

FIG. 6 shows a graph 600 of the total force (F total) observed using a balance plate for healthy 86 year old female (B) performing the same sit-to-stand task as in FIG. 2.

With regard to FIG. 2 subject (A) is reasonably fit and consequently the curve shown is illustrative of the performance expected in a situation of ‘low risk’. In comparison, FIG. 6 shows an 86 year old female with significant postural instability. As further discussed below, the curve in FIG. 6 shows several aspects that may be correlated with an increased and/or fall risk. For example it may be easily seen that the time to completion of the sit-to-stand task has significantly increased. That is, the time from the commencement of the rise in observed force to the time where the observed force substantially reaches its final level has increased. Additionally, may be noted that there is a considerable increase in high frequency components in the curve. This may be seen by the many additional oscillations of noticeable amplitude as the task is being performed and even after the task has been completed.

FIGS. 2 and 6 thus show a side by side comparison of the performance of the relatively stable and lower fall risk subject (A) and significantly unstable and thus higher fall risk subject (B). Further, these figures show the change in nature of task performance with respect to both shape of curve and time to complete.

Additionally, the shape of curves may be analyzed by using polynomial fit or other standard data reduction methods in order to allow quantitative comparison. For example, a curve requiring higher order polynomials, or relying to a greater degree on higher order polynomials may be correlated with an increased fall risk.

Further, it should be noted that in transitioning from one curve to the other there is a basic difference in the functional description that may generate an unambiguous indication of change in condition. As an example, calculation of the first derivative of a polynomial function fitted to the performance curve would indicate transition from an ‘overshoot’ to a gradual approach as shown explicitly in the data displayed.

Alternatively, it may be seen in FIG. 2 that the observed force curve initially overshoots the final value. Conversely, in FIG. 6, the curve only gradually rises to the final value. In one embodiment, the presence or absence of an initial overshoot may be correlated to a decreased fall risk. In addition to the presence or absence of an overshoot, the size of the overshoot may relate to fall risk in that a larger overshoot may correlate to a decreased fall risk. Additionally, the time to reach the overshoot may relate to fall risk in that a faster time to reach overshoot may correlate to a lesser fall risk.

Additionally, comparison of the data sets in FIGS. 2 and 6 shows significantly larger excursions at high (greater than 5 Hz) frequencies in FIG. 6. Thus, a power spectrum analysis of such data may provide relate to fall risk in that greater power in a higher frequency spectrum may be correlated to increased fall risk.

FIG. 7 shows graphs of the total force (Ftotal) observed using a force plate for a 53 year old female subject (A) 710, 76 year old female subject (C) 720, and 86 year old female subject (B) 730 performing a sit-to-stand task.

FIG. 7 shows further illustrations of inter-subject differences that reflect increased or decreased fall risk (53, 76, 86 year old individuals on a single trial of sit-to-stand task). As shown in FIG. 7, the sit-to-stand task shows one or more of the following with increasing age: additional time to max force (correlated with increased fall risk), additional high frequency components with increasing age (correlated with increased fall risk), takes longer to reach steady state (correlated with increased fall risk), more low frequency amplitude oscillations during the task (correlated with increased fall risk), as well as the additional correlations discussed above.

Additionally, the curve 730 can be observed to include an initial failure to rise smoothly to the standing position, as noted by the large amplitude oscillation near the initial force. Such an initial failure may also be correlated with an increased fall risk.

One or more embodiments of the present system and method of its use baseline or standard data to populate data bases for comparison and therefore an emergent property of the system is that it becomes increasingly useful as the data bases increase in size and segmentation by relevant (demographic or medical) subcategories becomes possible. This emergent property provides real time access to comparative data that is highly desirable.

As data bases grow in size, the effectiveness of the present system increases through the ability to identify performance bounds and correlate them with specific demographic or medical information. For example, a time series of strength measurements on a single subject is observational and of limited diagnostic utility; comparison against normative data may readily identify ‘outliers’ which merit further attention. For greater clarity, the same situation exists in relation to chemical analyses of (for example) blood where analyte concentrations require comparison to ‘reference values’ to be generally useful and where groups of analytes co-vary in a manner analogous to specific attributes of performance. Fall risk is known to be multivariate and significantly depends on ‘external’ factors which interact differently with subjects—trivially, for example, visual disturbances may affect balance differently from loss of strength.

The analysis and recommendations made are emergent properties of the system that result from its use with increasing population size and diversity. Analytical methods for dealing with large data sets are well known in present art as are techniques for presentation of such results.

Below are some additional examples of one or more embodiments of the present system:

### EXAMPLE 1

A subject stands on a horizontal ‘balance board’ (a plate with a number of load sensors so that shifts in force in the X-Y plane of the balance board may be detected; the time constant of the device should (preferably) allow data to be acquired at a frequency greater than 20 Hz. Once the subject is in position, tasks such as ‘maintain static position for 1 minute’ (although other time periods may be employed such as 3 s, 5 s, 10 s, 15 s, or 30 s) or ‘close your eyes and maintain static position for 1 minute’ (although other time periods may be employed such as 3 s, 5 s, 10 s, 15 s, or 30 s) or ‘with your eyes closed, lift your arms to horizontal in front of you and hold this position for 1 minute’ (although other time periods may be employed such as 3 s, 5 s, 10 s, 15 s, or 30 s) are performed and data from the load sensors is captured via a standard (digitizing) interface and transmitted wirelessly to a gateway.

The data stream may be immediately displayed to both the subject and the test operator if feedback is desired and simultaneously sent through a network to a computer or computers where it is entered into a database and then analyzed by comparison with performance data obtained through carrying out the same task.

Such analysis may compare frequency distribution of ‘excursions around mean values’ or aperiodic features such as lateral drift and requires for its interpretation comparison to other subjects, preferably with known demographic or other attributes. Further, the present system becomes increasingly effective with the development and use of data bases which record measurements as data sets associated with specific task performance and demographic data.

In another embodiment, the analysis may compare the data received with an idealized curve representing the desired characteristics for performance of the task. The idealized curve may be scaled based on one or more of the gender, weight, and age of the patient.

In another embodiment, a model has been formed that includes a list of curve characteristics that may be automatically searched for in the curve. Additionally, each curve characteristic may be evaluated for its impact on fall risk, for example increased fall risk, decreased fall risk, or no significant impact.

For example, a list of curve characteristics may include the presence of an overshoot, the amount of time to steady-state (or at least relatively stable state) in the performance of a sit-to-stand task, and the relative distribution of low and high frequency components present in the curve. Alternatively, any or all of the correlations mentioned herein may be included in the list of curve characteristics.

Additionally, the curve characteristics on the list may be reviewed, analyzed, and combined to form a composite fall risk score. In addition more quantitative measurements may be combined with the binary, present-or-not determinations, like the presence of an overshoot. For example, each of the three characteristics of the curve characteristic list may account for one third of a combined risk score. For example, the presence or absence of an overshoot of at least 5% from the final force value (alternatively any over shoot or an overshoot of 3%, 10%, or 20%) may provide one third of the composite score.

A second third may be found by comparing the time to stable state to an expected time. That is, if the rise to standing was accomplished in 2 seconds or less (alternatively 1 s, 1.5 s, 2.5 s, 3 s, 4 s, or 5 s), then the time-to-rise score is at its maximum. Alternatively, the score may be scaled down based on increasing time. For example, the score may be determined by dividing the expected rise time by the actual rise time so that an actual rise time or 4 s results in a 50% score.

The final third may be found by comparing the relative distribution of low and high frequency components present in the curve. Alternatively, the excursions of the force curve from the stable state may be reviewed for a determination of their relative low and high frequency components. For example, the ratio of the power at a frequency higher than a threshold to power at a frequency lower than a threshold may be employed. For example, the ratio of power at a frequency higher than 10 Hz (alternatively, 5 Hz, 15 Hz, 20 Hz, or 50 Hz) to power at a frequency less than 1 Hz (alternatively 0.5 Hz, 2 Hz, 3 Hz, or 4 Hz) may be employed as a score. For example, the ration may be 60% which would result in a 60% score.

The resulting three thirds may then be combined into a final score, for example by either addition or averaging. The final score may then be compared to a threshold for fall risk. For example, if the final score is less than 50%, s significant risk for a fall has been identified.

Alternatively, the final score may be compared to several thresholds, for example, lesser fall risk, moderate fall risk, and high fall risk thresholds. Each fall risk may be correlated with different actions to be taken to mediate the fall risk.

Additionally, the various aspects of the model mentioned above may be adjusted based on population-wide fall outcome data. For example, if an initial determination had been made that one-third of the score should be based on the time to steady state/completion, but analysis of population data indicates that the actual occurrence of falls is more heavily correlated with the time to rise, then the percentage of the total fall risk score that is attributable to the time to rise measurement may be increased, for example to 50%. Alternatively, if the factor is found to be less correlated to the actual occurrence of falls that take place, the percentage of the total risk score attributable to that factor may be reduced.

Further, additionally, the thresholds within a specific factor may also be adjusted based on the population-wide fall outcome data. For example, if an initial threshold of the time-to-rise factor was 2 s, but it was found through an analysis of fall outcome data that the use of a threshold of 3 s was more strongly correlated to and therefore predictive of fall outcome, then the threshold of 3 s may be employed. Additionally, when analyzing the fall outcome data, the most preferred threshold value may be other than a whole number.

### EXAMPLE 2

A balance board, as in the previous example, is wirelessly linked to a computer hosting data acquisition software and a user-guiding test protocol which defines specific tasks. On a signal or the instruction of a tester (such as a nurse) a subject attempts a sit-to-stand motion sequence starting with both feet on the balance board and ending with the subject in a stable standing position. (alternately a stand-to-sit task or other modified task may be specified). The data set for the test may be defined either from an arbitrary start time or alternately by a defined time series counting back from the end of the test sequence. The latter avoids start transients and is generally more easily determined in a hospital or clinical setting.

Several repeats of the defined task (sit-to-stand) may be performed. Immediate feedback from the sensor system is optional and generally helpful to both the patient and therapist since it provides verification of task performance.

Performance data sets are tagged with identifying information specific to the subject and session and transmitted through the (secure) network to a data repository and data analysis where using methods generally known in mathematics (signal extraction, averaging and statistical processing) relevant performance information may be extracted and compared to both population data and time series information for the particular subject. The ability to do quantitative comparisons of specific task performance data across defined populations as well as over time is a feature of the present system. It should be noted that such comparisons do not require an explicit biomechanical model of the task being performed. For example, in a sit-to-stand task, several features of the force-vs-time curve reflect inter-subject variations. With reference to the figures above and without limiting generality, features of the force-time graphs may be visually distinguished and provide specific opportunities to characterize and compare task performance. Examples within this set of graphs include (without limitation): duration of rise, overshoot and ‘settling’, low amplitude oscillations at relatively high frequency, and lateral asymmetry.

Other features of the graph may be identified and data may be analyzed using standard methods or algorithms developed for feature extraction. Note that, in general, data sets may be scaled or normalized for the purpose of comparison between subjects or repetitions by the same subject.

The general shape of the curve changes with physical condition in ways that reflect increased fall risk which may be a proxy for more general functional ability.

As an example, compare FIG. 7 which illustrates that the same task, performed by different individuals may produce significantly different performance data and fall risk. In this specific example the two subjects performed the same specified task but had different physical abilities correlated to their ages.

Such correlations may be established experimentally thus the inclusion of a data base of performances together with relevant demographic information is a desirable and distinguishing feature of the present system.

A general relationship exists between specific task performance and fall risk since specific task performance invokes multiple sub-activities; this allows performance comparison across populations by demographic or other segmentation criteria.

Data reduction (for example polynomial curve fitting and differentiation) may provide an indicator of fitness or strength (inversely related to fall risk). In comparison to a young and vigorous performance as shown in FIG. 7 there is a fundamental shape change in the force-vs-time graph for an elderly and somewhat infirm subject as shown in FIG. 7. Specifically, the curve 710 of FIG. 7 has a pronounced overshoot result of ‘bounce’ while the elderly subject's performance of the same task as shown in curve 730 indicates both a slower time to rise and a fundamentally different force-vs-time curve.

### EXAMPLE 3

In a practical (clinical) setting it is desirable to include information from a patient's musculo-skeletal evaluation record (including fall risk) in their medical record and to be able to record and annotate management and billing records. It is clear to a person skilled in computational management of data that such data may readily be passed back to or from the system described above and that such a system may be configured to the requirements (privacy and security) of medical records management. Accordingly in an embodiment of the present invention, the system permits access to and from medical and management data systems for reporting purposes and may be integrated with such systems.

Although only a few exemplary embodiments of this invention have been described above, those skilled in the art will readily appreciate that many other equivalents are possible without materially departing from the novel teachings and advantages presented herein. Accordingly, all such modifications are intended to be included within the scope of this invention.

Thus, one or more embodiments of the systems and methods described herein comprises several elements which in combination may measure attributes of physical performance which yield information relevant to assessing fall risk. Assessment of fall risk may not require an explicit biomechanical model.

In addition to the regions or aspects of the curves identified as relevant above, any of the following regions or aspects of the curves related to the performance of a task may be used for evaluation alone or in conjunction with others: A) Time from start of recording to initiating of action, B) Smoothness of rise (or slope), C) Presence of “Hitch” in rise, D) Time to rise from initiation to peak, E) Overshoot percentage from steady state, F) Speed of oscillation near steady state, G) Amplitude of oscillation at steady state, H) Time to steady state, I) Number of zero passes, and J) Concave-ness of curve.

Additionally, the curve for comparison with a patient's data may be an idealized curve, a composite of numerous healthy people, or may be broken out by age/sex cohort for comparison. Additionally, the thresholds identified above may be adaptive for age/sex cohort comparison.

Additionally, the testing protocols may include any of several options. For example, the testing Protocol may call for performing more than a single measurement to provide data for analysis. For example, the Protocol may direct the patient to perform the same measurement type multiple times and average/interpolate results for analysis. Alternatively, the Protocol may: direct the patient to perform two or more different tasks in a set pattern, direct the patient to perform two or more different tasks in an adaptive pattern (wherein the second or subsequent measurements depend on the results of preceding measurements), when two or more different tasks are performed, present/analyze results separately; and/or when two or more different types of measurements are performed, combine the results of both for analysis.

Additionally, as mentioned above, the central server may allow repository linking. For example, test results for a specific patient may be linked with previous test results, and/or medical and billing information for the client. The availability of the previously stored test results may allow the system to provide tending and/or tracking of fall risk information for the patient. Additionally, the medical information may include information such as surgeries or medication so that their impact may be considered when analyzing fall risk.

Also, one or more embodiment of the present system may be integrated with other systems commonly used in the practice of medicine, such as accounting systems, insurance management systems, reporting of recommendations, therapy, and/or results to the patient's primary car physician, and reporting and/or website access to results by the patient over the internet.

Additionally, it has been noted that many hospitals or other medical facilities often over-estimate fall risk. This typically results in the hospital providing unnecessary services towards care. Such services represent excessive costs for no benefit.

For example, if a hospital possessed the ability to accurately assess the fall risk of an individual as they rise from a hospital bed, then (assuming that the fall risk is minimal) then the hospital may choose to forgo certain practices and allocate resources differently.

For example, a patient who is classified at high risk requires more intensive monitoring than a patient who is classified at lower risk. This typically involves aspects such as more nursing time and/or different appliances. For example for some acute care hospitals, the annualized fall risk per admission may be as high as 5%. However, in such institutions, 2-5 times that number of the patients may be labeled as high risk for fall which may cause the expenditure of substantial resources that may not be appropriately deployed.

Additionally, one or more embodiments of the present system and method may provide the ability to make a finer judgment as to the fall risk than is currently available. For example, many hospitals engage in a “gut” determination or “educated guess” determination. Conversely, one or more embodiments of the present invention may provide a much more consistent, repeatable, and accurate determination for fall risk. Further, the determination may become even better with increasing analysis of population-wide fall risk outcomes and the adaptation of the risk determination model in light of the statistical realities that become apparent.

Additionally, one or more embodiments of the present system may provide a legitimate, defensible reason why a hospital chose to provide or not provide a certain level of fall risk management. Such a system may be especially useful in the case of a fall resulting in an injury, especially where such an even may result in litigation. In such a circumstance, the judgment of the trained or self-taught professional that made the fall risk determination may be challenged or called into question. For example, if the fall risk evaluator made a determination that fall risk was unlikely, but a fall still occurred, it may be very attractive to a jury to view the evaluator's judgment as flawed and consequently place blame for the fall on the evaluator. Conversely, many juries do not apply similar reasoning when the evaluation comes from a machine. In such a situation, where a fall risk was determined by a machine to be low and a fall still occurred, a jury may be more likely to choose to acknowledge the reality that no predictive model is completely perfect and forgo or minimize the imposition of liability. Alternatively, the fall risk evaluation made by the machine (assuming it corresponds to the fall risk determination made by the evaluator) may serve to restore the credibility of the evaluator in the eye of the jury.

As mentioned above, it is noted that many factors may be considered in arriving an overall fall risk assessment for a specific patient at a specific time at a specific facility under a specific set of conditions. For example, the condition of the physical plant of the hospital, the additional medications taken by the patient, or an overall assessment of the patient's judgment may be very relevant to the determination of an overall fall risk assessment. However, one or more embodiments of the present invention may provide insight into the physical condition of the patient which is a very significant factor in the determination of the overall fall risk assessment.

While particular elements, embodiments, and applications of the present invention have been shown and described, it is understood that the invention is not limited thereto because modifications may be made by those skilled in the art, particularly in light of the foregoing teaching. It is therefore contemplated by the appended claims to cover such modifications and incorporate those features which come within the spirit and scope of the invention.

1. A system for assessment of patient fall risk.
